(19) United States
(12) Patent Application Publication
(10) Pub. No.: US 2022/0273254 A1
(43) Pub. Date: Sep. 1, 2022

(54) X-RAY IMAGING DEVICE AND ASSOCIATED IMAGING METHOD

(71) Applicants: UNIVERSITÉ GRENOBLE ALPES, SAINT-MARTIN-D'HÉRES (FR); INSERM-INSTITUT NATIONAL DE LA SANTÉ ET DE LA RECHERCHE MÉDICALE, PARIS (FR); NOVITOM, GRENOBLE (FR); ESRF European Synchrotron Radiation Facility, GRENOBLE (FR)

(72) Inventors: Hélène LABRIET, GRENOBLE (FR); Sébastien BERUJON, GRENOBLE (FR); Emmanuel BRUN, GRENOBLE (FR)

(21) Appl. No.: 17/625,701

(22) PCT Filed: Jun. 30, 2020
(86) PCT No.: PCT/FR2020/051140

§ 371 Date: Jan. 7, 2022

(30) Foreign Application Priority Data
Jul. 11, 2019 (FR) 1907827

(51) Int. Cl. A61B 6/00; G01N 23/046; G01N 23/041

(52) U.S. Cl. CPC A61B 6/484; A61B 6/5217; A61B 6/4035; A61B 6/502; G01N 23/046; G01N 23/041; G01N 2223/401

(57) ABSTRACT

The present invention relates to an X-ray imaging device having a simplified architecture and being easily adaptable to conventional X-ray sources, as well as to an imaging method associated with this imaging device.

The present invention relates generally to an X-ray imaging device using a simplified architecture, the device being able to be applied mainly to the field of medical imaging, but also to that of the X-ray imaging of material characterisation, or in the field of security (for example for checking luggage at airports).

The phase contrast imaging has revolutionised the X-ray imaging for twenty years and has allowed creating contrasts in materials, known to be transparent to X-rays, by measuring the phase change of X-rays, due to the refraction of X-rays during the passage of the sample^{[1] [2] [3]}. Indeed, this technique uses information concerning the changes in the phase of an X-ray beam which passes through an object, in order to obtain an image of this object.

Unlike conventional x-ray imaging techniques, which measure the attenuation of the intensity of the x-ray beam, the phase contrast imaging indirectly measures the phase shift caused by the sample, the latter being transformed into a variation of intensity measurable by an X-ray detector. This type of imaging is widely used with synchrotron sources^{[4] [5] [6] [7]}.

Among the traditional X-ray sources, the sources for computer-assisted microtomography are known in particular, for example of the nanoFocus or microFocus type, the sources for a brightness amplifier type apparatus, the sources for a mammography type apparatus, and the radiography type sources, and computed tomography sources. The interest of traditional X-ray sources relative to sources such as a synchrotron source lies in their cost, less expensive, their size, more compact and their greater ease of handling.

Different phase contrast X-ray imaging devices using these conventional X-ray sources ^{[13] [14] [15]} are known. However, these devices are complex, require X-ray sources with high spatial coherence, or even involve significant radiation doses for the formation of the images. In particular, they require a great mechanical stability, which is performed to the detriment of the width of the field of view or of the level of resolution of the obtained images.

There is therefore a need to have phase contrast X-ray imaging devices which are easy to implement and which can use conventional sources as X-ray sources.

The technical problem which the invention proposes to solve is thus both to simplify the technical implementation of phase contrast X-ray imaging, and to make it more flexible, by the possibility of using a wider range of X-ray sources, including conventional sources.

In order to solve this problem while overcoming the previously mentioned drawbacks, the applicant has developed an X-ray imaging device, in particular in phase contrast, comprising:
an X-ray source,
a spatial intensity modulator, having a maximum thickness and a minimum thickness, capable of being passed through by an X-ray beam from the X-ray source, and of forming an X-ray beam which is spatially intensity modulated,
a sample support, capable of supporting a sample, said sample being intended to be passed through by at least one portion of said spatially intensity modulated X-ray beam and transmitting a refracted spatially intensity modulated X-ray beam, the sample, when it is on said support, being located at a distance d from the X-ray source,
an X-ray detection system, located at a distance D from the X-ray source, and comprising a two-dimensional X-ray sensor provided with a plurality of photo-detector elements, each having the same given size, said detection system being capable, in a first configuration of the device in which the device does not comprise a sample, of detecting a first X-ray beam directly from the spatial intensity modulator, said first X-ray beam directly from the spatial intensity modulator having a first intensity modulation, and of transforming said first beam into a first electrical signal, and, in a second configuration of the device in which a sample is disposed on the sample support, of detecting a second X-ray beam, passing through the spatial intensity modulator then the sample, said second X-ray beam refracted by the sample having a second intensity modulation, and of transforming said second beam into a second electrical signal,
an electronic processing unit, capable of receiving the first electrical signal and of processing it so as to generate a first image comprising a plurality of pixels, each having the same given size, and of receiving the second electrical signal and of processing it so as to generate a second image, and capable of generating, from said first image and from said second image, at least one characteristic image of said sample,

said imaging device being characterised in that the difference between the maximum thickness and the minimum thickness of the spatial intensity modulator, called average roughness, is comprised between two and twenty times the size of the pixels of the first image, said size being equal to the product of the size of the photo-detector elements by the ratio d/D.

The specific choice of the average roughness of the spatial intensity modulator allows adjusting, in the first image, the first intensity modulation of the first X-ray beam directly from the spatial intensity modulator. Thus, this choice allows optimising the quality of said at least one characteristic image of said sample.

Preferably, the average roughness of the spatial intensity modulator is comprised between two and fifteen times the size of the pixels of the first image.

More preferably, the average roughness of the spatial intensity modulator is comprised between two and ten times the size of the pixels of the first image.

More preferably, the average roughness of the spatial intensity modulator is comprised between three and seven times the size of the pixels of the first image.

In the case where the average roughness of the spatial intensity modulator is less than twice the size of the pixels of the first image, the drawback is that it is difficult to distinguish in the first and the second image the intensity modulation of the X-ray beam of the inherent photonic noise in the first and second X-ray beams.

In the case where the average roughness of the spatial intensity modulator is greater than twenty times the size of the pixels of the first image, the drawback is that said at least one characteristic image of said sample has poor resolution.

The photo-detector elements can be of the same square shape and each have the same given size equal to the length of the side of the square shape.

The photo-detector elements can be of the same rectangular shape and each have the same given size equal to the length of the rectangular shape.

The photo-detector elements can be of the same hexagonal shape and each have the same given size equal to the distance separating two vertices of the hexagonal shape which are diametrically opposed.

Advantageously, the electronic processing unit is capable of generating said at least one characteristic image of said sample, from the difference between said first image and said second image.

The electronic processing unit can be capable of generate said at least one characteristic image of said sample depending on the gradient of the phase of the refracted X-ray beam received by the X-ray detection system in the second configuration of the device.

Preferably, the spatial intensity modulator comprises an element selected from a metal, a metalloid, a light element and the mixtures thereof, the atomic number of said element being comprised between 13 and 80.

More particularly, the element of the spatial intensity modulator can be selected from aluminium, silicon, iron, copper, titanium, nickel, silver, tin, gold, and the mixtures thereof.

In one embodiment of the invention, the spatial intensity modulator (3) comprises powder and/or particles.

In another embodiment of the invention, the spatial intensity modulator is manufactured by three-dimensional printing, abrasion, or moulding.

Advantageously, the X-ray source has an energy comprised between 10 and 300 keV. This energy can be obtained by using an X-ray tube capable of accelerating electrons between two electrodes having a peak potential difference comprised between 10 and 300 kVp.

Preferably, the X-ray source has an energy comprised between 20 and 300 keV.

Again preferably, the X-ray source has an energy comprised between 20 and 180 keV.

Again preferably, the X-ray source has an energy comprised between 20 and 120 keV.

Again preferably, the X-ray source has an energy comprised between 15 and 120 keV.

Advantageously, the X-ray source is selected from a source for computer-assisted microtomography, for example of the nanoFocus or microFocus type, a source for a brightness amplifier type apparatus, a source for a mammography type apparatus, and a radiography type source.

Advantageously, the material of the spatial intensity modulator can be selected depending on the energy of the X-rays emitted by the X-ray source.

In particular, the material of the spatial intensity modulator can be selected so that its visibility, equal to the ratio of the standard deviation of the distribution of the intensities of the pixels of the first image I_{ref}(x, y) on the average of the intensities of the pixels of the first image I_{ref}(x, y), namely comprised between 0.02 and 0.30.

Preferably, the material of the spatial intensity modulator is selected so that its visibility is comprised between 0.05 and 0.20.

In the case where the visibility of the spatial intensity modulator is greater than 0.30, the drawback is that the characteristic image of the sample (E) has reduced dynamics.

In the case where the X-ray source has an energy comprised between 15 and 40 keV, the material of the spatial intensity modulator can be selected from silicon, titanium and aluminium.

In the case where the X-ray source has an energy comprised between 40 and 100 keV, the material of the spatial intensity modulator can be selected from copper, iron, cobalt and nickel.

In the case where the X-ray source has an energy comprised between 100 and 120 keV, the material of the spatial intensity modulator can be selected from silver, zinc, tin and molybdenum.

In the case where the X-ray source has an energy greater than 120 keV, the material of the spatial intensity modulator can be selected from gold and tungsten.

The underlying advantage of the choice of the material forming the spatial intensity modulator is to allow the use of x-ray sources of different energies, in particular high energies, which it is currently difficult to use, and a wide range of image resolutions.

Advantageously, it is possible to have several spatial intensity modulators consecutively in the direction of propagation of the X-ray beam from the X-ray source.

In a first embodiment, the sample support is rotatably mounted about an axis of rotation orthogonal to the main direction of the X-ray beam from the X-ray source, to allow the rotation of the sample around the x-ray source.

In a second embodiment, the assembly formed by the X-ray source and the X-ray detection system is rotatably mounted around the sample support, to allow the rotation of the assembly formed by the X-ray source and the X-ray detection system is rotatably mounted around the sample.

The present invention also relates to an X-ray imaging method, implementing an X-ray imaging device as previously described.

In a first embodiment of the imaging method according to the invention, said method is a two-dimensional imaging method, said method comprising the following steps:

a/ exposing the spatial intensity modulator to an X-ray beam from the X-ray source,

b/ in the first configuration of the device, detecting and transforming the first X-ray beam into a first electrical signal,

c/ receiving and processing, by the electronic processing unit, the first electrical signal, so as to generate at least one first image,

d/ in the second configuration of the device, detecting and transforming the second X-ray beam into a second electrical signal,

e/ receiving and processing by the electronic processing unit, the second electrical signal, so as to generate at least one second image,

f/ generating, by the electronic processing unit, from said at least one first image and said at least one second image, at least one image selected from a transmission image, an image of the gradient of the differential phase in two directions which are orthogonal and parallel to a plane perpendicular to the main direction of the X-ray beam from the X-ray source, an image of the phase, and an image of the scattering of the sample.

In a second embodiment of the imaging method according to the invention, in the case where the sample support is rotatably mounted about an axis of rotation orthogonal to the main direction of the X-rays from the X-ray source, or in the case where the assembly formed by the X-ray source and the X-ray detection system is rotatably mounted around the sample support, the imaging method is a three-dimensional imaging method, comprising the following steps:

a/ exposing the spatial intensity modulator to an X-ray beam from the X-ray source,

b/ in the first configuration of the device, detecting and transforming the first X-ray beam into a first electrical signal,

c/ receiving and processing, by the electronic processing unit, the first electrical signal, so as to generate at least one first image,

d/ in the second configuration of the device, in the case where either the sample support is rotatably mounted about an axis of rotation orthogonal to the main direction of the X-ray beam from the X-ray source, or the assembly formed by the X-ray source and the X-ray detection system is rotatably mounted around the sample support, detecting the second X-ray beam and transforming it into a second electrical signal, for N given positions (1, . . . , i, . . . , N), the N given positions being either positions of the sample, each corresponding to a given rotation of the sample support, or positions of the assembly formed by the X-ray source and the X-ray detection system,

e/ receiving and processing by the electronic processing unit, for each of the N positions, the second electrical signal, so as to generate for each of the N positions at least one second image,

f/ generating, by the electronic processing unit, from said at least one first image and all second images, at least one image selected from a three-dimensional transmission image, a three-dimensional image of the gradient of the differential phase in two directions which are orthogonal and parallel to a plane perpendicular to the main direction of the X-rays from the X-ray source, a three-dimensional image of the phase, and a three-dimensional image of the scattering of the sample.

Advantageously, a plurality of first images and a plurality of second images are generated and are combined to generate by the electronic processing unit at least one two-dimensional or three-dimensional image selected from a transmission image, an image of the phase gradient in two directions which are orthogonal and parallel to a plane perpendicular to the main direction of the X-ray beam from the X-ray source, an image of the phase, and an image of the scattering of the sample.

In a third embodiment of the imaging method according to the invention, the method is a method for time tracking structures contained in a sample, during a time interval T, comprising the following steps:

a/ exposing the spatial intensity modulator to an X-ray beam from the X-ray source,

b/ in the first configuration of the device, detecting and transforming the first X-ray beam into a first electrical signal,

c/ receiving and processing, by the electronic processing unit, the first electrical signal, so as to generate at least one first image,

d/ detecting, fora plurality of N instants ti, i being an integer comprised between 1 and N, contained in the time interval T, and transforming the second X-ray beam into a plurality of N second electrical signals,

e/ receiving and processing, by the electronic processing unit, the N second electrical signals, so as to generate N second images,

f/ generating, by the electronic processing unit, from said at least one first image and N second images, a sequence of N successive images, said N successive images being transmission images, gradient images of the differential phase in two directions which are orthogonal and parallel to a plane perpendicular to the main direction of the X-rays from the x-ray source, images of the phase, or images of the scattering of the sample.

Advantageously, for this third embodiment of the imaging method according to the invention, a plurality of first images and a plurality of second images are generated and are combined to generate by the electronic processing unit at least one sequence of N successive images, said N successive images being transmission images, gradient images of the differential phase in two directions which are orthogonal and parallel to a plane perpendicular to the main direction of the X-rays from the X-ray source, images of the phase, or images of the scattering of the sample

In this third embodiment of the imaging method according to the invention, the latter may further comprise a step of processing at least one sequence of N successive images to obtain elastographic data of the structures of the sample.

The general advantage of the invention is to simplify an X-ray imaging device, as well as the associated imaging process, by resorting to conventional X-ray sources and to a material which is easy to manufacture and to implement, in particular to optimised spatial intensity modulators. Thus, thanks to the specific choice of the average roughness of the spatial intensity modulator, it is allowed to implement, in the device, a wide variety of conventional X-ray sources, in particular at high energies, and to obtain a wide range of resolutions of the obtained images. It is possible, in particular, to improve the level of detail visible on the images obtained by the device, with conventional X-ray sources. Thanks to the invention, it is possible to implement a phase contrast imaging method using conventional sources and to increase the contrast of the obtained image. The aspects which are simple, inexpensive, and adaptable to the laboratory sources of the invention allow a current use thereof. The implementation of the device with medical X-ray sources thus makes possible the three-dimensional imaging of patients.

Other advantages and features of the present invention will result from the following description, given by way of non-limiting example and made with reference to the appended figures:

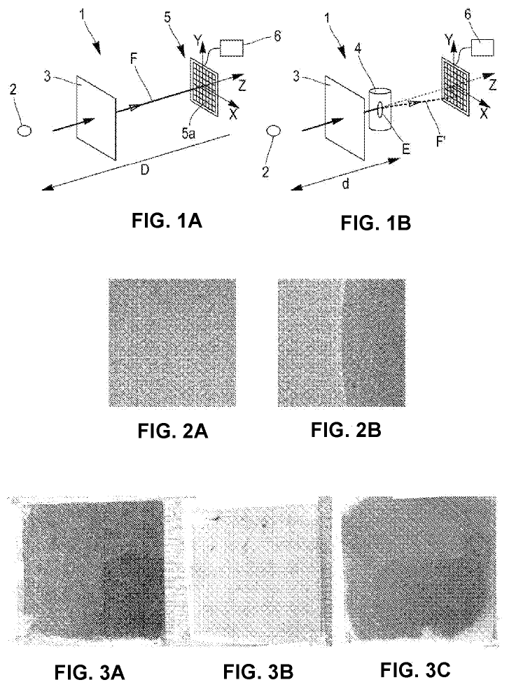

FIG. 1 schematically illustrates an X-ray imaging device according to the invention in a first and a second configuration.

FIG. 2 shows two images which can be obtained with respectively the first and the second configuration of the device according to the invention.

FIG. 3 shows different top view photographs of a first type of spatial intensity modulators that can be used in the device according to the invention.

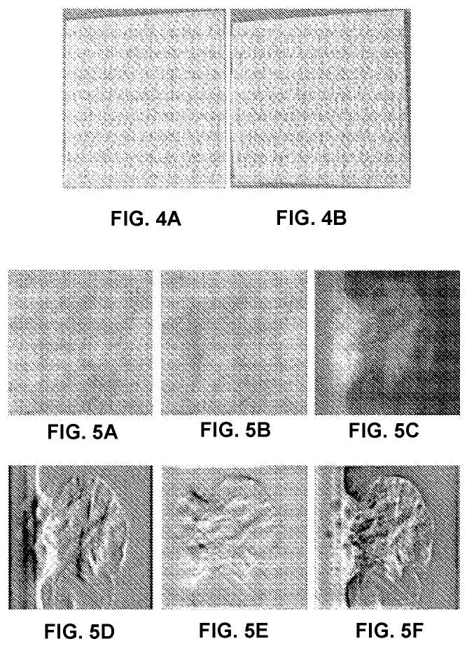

FIG. 4 shows different top view photographs of a second type of spatial intensity modulators that can be used in the device according to the invention.

FIG. 5 shows a series of images obtained according to a first embodiment of the imaging method according to the invention, with a first type of spatial intensity modulator.

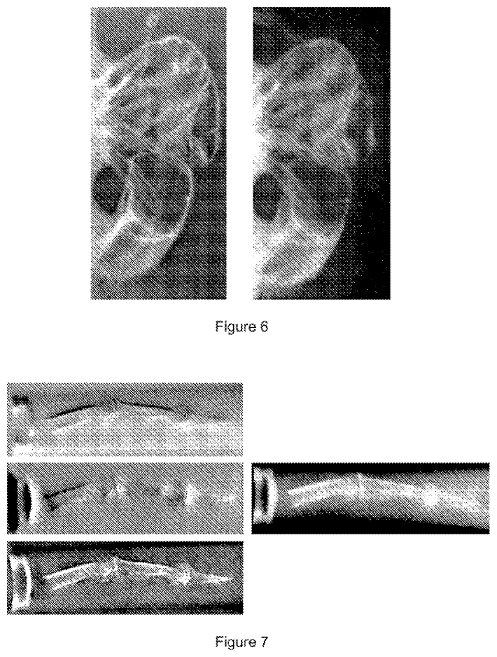

FIG. 6 shows two images of the same sample obtained with two different X-rays source-spatial intensity modulator pairs.

FIG. 7 shows different images of a finger which are obtained according to a first embodiment of the imaging method according to the invention, with a second type of spatial intensity modulator, and a conventional radiography of the finger.

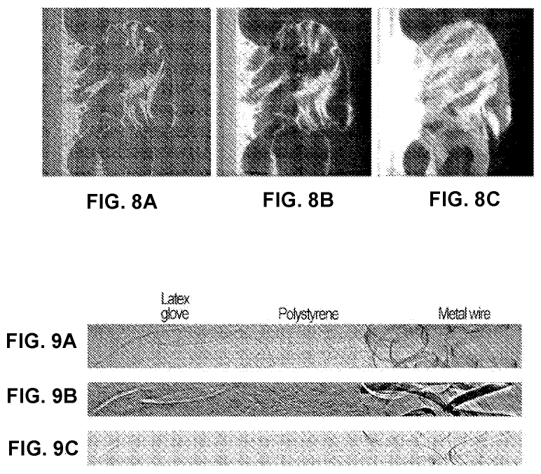

FIG. 8 shows a conventional radiography obtained by computer-assisted tomography of a sample, a two-dimensional image of the same sample obtained with a second embodiment of the imaging method according to the invention, with a first type of spatial intensity modulator, and a volume rendering image of the same sample obtained according to a second embodiment of the imaging method according to the invention.

FIG. 9 shows a series of images of a sample composed of a latex glove, a sample of polystyrene and a metal wire, obtained according to a third embodiment of the imaging method according to invention, with an application to elastography.

FIG. 1 schematically illustrates an X-ray imaging device 1 according to the invention in two different configurations, without a sample to be imaged, as represented in the portion (a) of FIG. 1, and with a sample E to be imaged, as shown in the portion (b) of FIG. 1.

In a first configuration (a) of the X-ray imaging device 1 according to the invention, without sample E to be imaged, an X-ray source 2 illuminates a spatial intensity modulator 3. The term “spatial intensity modulator” means an object of substantially planar shape, and having a thickness, allowing, through the spatial structure thereof, to spatially modulate the intensity of an X-ray beam F which passes therethrough. After passing through the spatial intensity modulator 3, the X-ray beam F has a spatial intensity modulation. An X-ray detection system 5, located behind the spatial intensity modulator 3, and at a distance D from the X-ray source 2, detects this X-ray beam F directly from the spatial intensity modulator 3, and transforms this first beam into a first electrical signal S_{elec_ref}. The distance D is measured along the axis orthogonal to the spatial intensity modulator 3 and to the X-ray detection system 5 and passing through the X-ray source 2. Moreover, the x-ray detection system 5 comprises a two-dimensional X-ray sensor provided with a plurality of photo-detector elements 5a, each having the same given size, each of the photo-detector elements 5a receiving a portion of the X-ray beam F. Each of the photo-detector elements 5a may be square or rectangular in shape, the size of the photo-detector element 5a designating either the length of the side of the square or either of the length or width of the rectangle. An electronic processing unit 6 is used to receive the first electrical signal S_{elec_ref} and process it so as to generate a first image I_{ref}(x, y) comprising a plurality of pixels, each having the same given size. An example of the first image I_{ref}(x, y) is shown in image (a) of FIG. 2.

In a second configuration (b) of the X-ray imaging device 1 according to the invention, a sample E to be imaged is positioned on a sample support 4, positioned downstream of the spatial intensity modulator 3, at a distance d from the X-ray source 2. The distance d is measured along the axis z orthogonal to the spatial intensity modulator 3 and to the x-ray detection system 5, and passing through the X-ray source 2 When the X-ray source 2 illuminates the spatial intensity modulator 3, at least one portion of the spatially intensity modulated X-ray beam F from the spatial intensity modulator 3 passes through the sample E. The latter refracts a spatially intensity modulated X-ray beam F′, of different modulation relative to the first X-ray beam F directly from the spatial intensity modulator 3, due to the refraction that it induces on this first beam. The term “refraction” means the deviation relative to the axis z of the rays, due to the local variations in refractive index encountered thereby when crossing the sample E. In the portion (a) of FIG. 1, illustrating the first configuration of the X-ray imaging device 1 according to the invention, without sample E, a reference beam F is shown schematically in solid line, which is not refracted. In the portion (b) of FIG. 1, illustrating the second configuration of the X-ray imaging device 1 according to the invention, with a sample E to be imaged, a refracted beam F′ following passage through the sample E is shown schematically in dashed line.

As in the first configuration of the device 1, the X-ray detection system 5 detects the refracted beam F′, which has passed through the spatial intensity modulator 3 then the sample E, and transforms it into a second electrical signal S_{elec_sample}. The electronic processing unit 6 is used to receive the second electrical signal S_{elec_sample} and process it so as to generate a second image Iₛₐₘₚₗₑ(x, y). An example of a second image Iₛₐₘₚₗₑ(x, y) is the image (b) in FIG. 2.

From a first image I_{ref}(x, y) obtained in the first configuration, without sample E to be imaged, and from a second image Iₛₐₘₚₗₑ(x, y) in the second configuration, with a sample E to be imaged, the electronic processing unit 6 can generate at least one characteristic image of the sample E. In this regard, the X-ray imaging device 1 according to the invention is a speckle imaging device. The speckle imaging devices implement a spatial modulation of the intensity of a beam of rays passing through a spatial intensity modulator in order to image a sample placed downstream of the spatial intensity modulator. The spatial intensity modulator can be in the form of a random object^{[8] [12]}. Traditional examples of random objects are sandpaper, biological filters, silica or steel wool.

The electronic processing unit 6 can generate at least one characteristic image of the sample E from the difference between a first image I_{ref}(x, y) and a second image Iₛₐₘₚₗₑ(x, y). The image resulting from the subtraction Iₛₐₘₚₗₑ(x, y)−I_{ref}(x, y) represents a direct measure of the refraction of the sample. In this image, an intensity modulation is present only in the places of the image where the sample E is present. An example of such an image Iₛₐₘₚₗₑ(x, y)−I_{ref}(x, y) is illustrated in FIG. 5 (c) which will be described later. The image resulting from the subtraction Iₛₐₘₚₗₑ(x, y)−I_{ref}(x, y) can indeed be subsequently processed by the electronic processing unit 6 to obtain one or more final images of the phase of the sample, by phase retrieval methods.

For example, digital processing methods, and based on the digital image correlation (DIC), can be implemented. The tracking of speckles (“X-ray Speckle Vector Tracking (XSVT)”, “X-ray-Speckle Scanning (XSS)”) is an example of this type of methods. These methods use in particular techniques of maximisation of cross-correlation^{[9]}, or of unified modulated pattern analysis (UMPA). In particular, the UMPA technique is based on a model of the interference pattern created by the sample^{[16]}.

In particular, the electronic processing unit 6 can generate at least one characteristic image of the sample E depending on the phase gradient of the refracted X-ray beam F′ received by the detection system 5 in the second device configuration (1). The images (d) and (e) of FIG. 5 are the images of the phase gradient of the X-ray beam F′ refracted by the sample E along the horizontal x and vertical y directions. The images of the phase gradient along the horizontal x and vertical y directions can then be processed by the electronic processing unit 6 to obtain one or more final images of the phase of the sample, for example by digital processing methods based on the principle of conservation of the flow. An example of these methods is the technique called “optical flow” ^{[17]}.

The spatial intensity modulator 3 has an average roughness, defined by the difference between the maximum thickness and the minimum thickness of the spatial intensity modulator. In accordance with the invention, the average roughness is selected so as to be comprised between two and twenty times the size of the pixels of the first image I_{ref}(x, y), in order to optimise the quality of the characteristic image of the sample E. The pixel size of the first image I_{ref}(x, y) is equal to the product of the size of the photo-detector elements 5a of the X-ray detection system 5 by the ratio D/d.

Thus, the X-ray imaging device 1 according to the invention is a device requiring little material and easy to use.

### EXAMPLES

### Example 1: Manufacture of Spatial Intensity Modulators

FIGS. 3 and 4 show several photographs of spatial intensity modulators 3 which can be used in an X-ray imaging device 1 according to the invention, according to two embodiments of the modulators.

FIG. 3 shows several photographs of spatial intensity modulators 3 according to a first embodiment. These spatial intensity modulators 3 have been designed using metal powders disposed on PMMA plates. Photography (a) shows a spatial intensity modulator 3 made from iron powder with an average particle size of 300 microns. This spatial intensity modulator thus has an average roughness of 90 microns. Photograph (b) shows a spatial intensity modulator 3 made from copper powder with an average particle size of 300 microns. This spatial intensity modulator thus has an average roughness of 300 microns. Photograph (c) shows a spatial intensity modulator 3 made from copper powder with an average particle size of 36 microns. This spatial intensity modulator thus has an average roughness of 36 microns. Thus, from a powder with a given average particle size, it is possible to manufacture a spatial intensity modulator according to a first embodiment of the invention.

FIG. 4 shows photographs of spatial intensity modulators 3 according to a second embodiment. These spatial intensity modulators 3 were manufactured by three-dimensional printing of materials marketed under the names PLA®, CopperFill® and BronzeFill® by the company Colorfabb. The geometric profile of these spatial intensity modulators 3 have been modelled by generating, for each of the spatial intensity modulators 3, a grayscale image, the grayscale representing the thickness in microns of the spatial intensity modulator 3. The grayscale images were obtained by generating a noise defined by an average value and a standard deviation (also called dispersion). In the case of the spatial intensity modulator 3 of the image (a) of FIG. 4, the average value and the standard deviation are 300 microns and 150 microns. The average roughness is 300 microns. In the case of the spatial intensity modulator 3 of the image (b) of FIG. 4, the average value and the standard deviation are 500 microns and 250 microns. The average roughness is 250 microns.

The above-mentioned methods for manufacturing spatial intensity modulator 3 thus make it possible to manufacture a wide range of spatial intensity modulators 3, allowing adapting them to a wide variety of X-ray imaging devices 1 according to the invention. The geometric features of the spatial intensity modulators, for example, the average particle size of a powder, or the average value and standard deviation of the noise of a grayscale image used in the model of the geometric profile of a spatial intensity modulator 3, are determined by the type of used sources and the desired image resolution. These methods for manufacturing spatial intensity modulators 3 which can be used within the framework of the invention are only examples and other methods can be considered, such as abrasion or moulding.

### Example 2: Two-Dimensional Image of a Fly

In this example, a first embodiment of the X-ray imaging method is implemented according to the invention, which is a two-dimensional imaging method, to image a sample consisting of a fly.

The used X-ray source 2 is a source for X-ray microtomography marketed under the name EasyTom XL® by the company Rx Solutions. The used spatial intensity modulator 3 consists of a copper powder with an average particle size of 45 microns placed between two PMMA plates. The average roughness of this modulator is 45 microns. The X-ray source 2 illuminates the sample supported by a sample support 4. An X-ray detection system 5 coupled to an electronic processing unit 6 allows generating a first image I_{ref}(x, y) in the first configuration of the X-ray imaging device 1 according to the invention, in which the device 1 does not include a sample E. The X-ray detection system 5 is placed at a distance D equal to 560 mm from the X-ray source 2. The sample E is placed at a distance d equal to 27 mm from the X-ray source. The photo-detector elements 5a of the X-ray detection system 5 are square in shape and have a physical size of 127 μm. The X-ray detection system 5 coupled to the electronic processing unit 6 then allows generating a second image Iₛₐₘₚₗₑ(x, y) obtained in the second configuration of the X-ray imaging device 1, in which the latter includes the sample to be observed E. The average roughness of the spatial intensity modulator 3 is in this case equal to 7.3 times the size of the pixels of the first image I_{ref}(x, y).

FIG. 5 shows a series of images obtained according to this first embodiment of the X-ray imaging method according to the invention. The images (a) and (b) illustrate respectively the image I_{ref}(x, y) and the image Iₛₐₘₚₗₑ(x, y). The image (c) illustrates the difference between the image I_{ref}(x, y) and the image Iₛₐₘₚₗₑ(x, y). The images (d) and (e) illustrate the gradient of the differential phase of the X-ray beam F′ refracted by the sample E in two directions which are orthogonal to each other and parallel to a plane perpendicular to the main direction of the X-rays. The image (f) represents an image of the phase of the sample E, reconstructed by processing, by the processing unit 6, of the images (d) and (e) of the gradient of the differential phase.

FIG. 6 shows a comparison between an image (a) of the same sample E (fly) obtained by phase contrast with an X-ray imaging device using a synchrotron type X-ray source and sandpaper as a spatial intensity modulator, and an image (b) of the phase of the X-ray beam F′ refracted by the sample E, obtained with the X-ray imaging device 1 according to the previously described invention. The image (b) has been corrected by a magnification factor, in order to compare the images (a) and (b) with a similar size of the sample E. A synchrotron type source is a powerful X-ray source of produced by high-energy electrons accelerated by electromagnetic waves and circulating in a storage ring. The contrast of the image (b) is higher than that of the image (a).

### Example 3: Two-Dimensional Image of a Finger

In this example, the first embodiment of the imaging method is implemented according to the invention, which is a two-dimensional imaging method, to image a sample consisting of a finger.

The X-ray imaging device 1 according to the invention herein includes an X-ray source 2 of the source type for a movable C-arm marketed under the name Arcadis Avantic® by the company Siemens, as well as a spatial intensity modulator consisting of a 3D printed membrane based on the material marketed under the name PLA® by the company Colorfabb. The average roughness of this modulator is 80 microns. The X-ray detection system 5 is placed at a distance D equal to 100 cm from the X-ray source 2. The sample E is placed at a distance d equal to 32 cm from the X-ray source. The photo-detector elements 5a of the x-ray detection system 5 are square in shape and have a size of 225 microns. The average roughness of the spatial intensity modulator 3 is in this case equal to 1.1 times the size of the pixels of the first image I_{ref}(x, y).

FIG. 7 shows a series of images of a sample E consisting of a finger which can be obtained with the imaging device described above. The image (a) is an image of the refraction in the horizontal direction x, which is proportional to the gradient of the phase of the X-ray beam F′ refracted by the sample E in this direction, in a plane perpendicular to the main direction of the rays from the X-ray source 2. The image (b) is an image of the refraction in the vertical direction y, which is proportional to the gradient of the phase of the X-ray beam F′ refracted by the sample E in this direction, in a plane perpendicular to the main direction of the rays from the X-ray source 2. The image (c) is an image of the phase of the X-ray beam F′ refracted by the sample E, calculated by digital integration of the images (a) and (b). The image (d) shows, for comparison, a conventional radiograph of the finger. A conventional radiograph is a picture of one or more anatomical structures resulting from the exposure to an X-ray beam of this or these anatomical structure(s) without a spatial intensity modulator, where the black areas correspond to air and the white areas correspond to bone structures. It can be observed that the quality of the image (c) is at least as good as that of the image (d), with in particular a better contrast and an absence of diffusion cone on the right portion of the image, while using a conventional x-ray source.

### Example 4: Three-Dimensional Image of a Fly

In this example, the second embodiment of the imaging method is implemented according to the invention, which is a three-dimensional imaging method, to image the sample E consisting of the fly of Example 2.

The used equipment corresponds to that used for obtaining the images of FIG. 5, with as X-ray source 2 a source for X-ray microtomography marketed under the name EasyTom XL® by the company Rx Solutions and as spatial intensity modulator 3 a spatial intensity modulator made of copper powder with an average particle size of 45 microns. The average roughness of this spatial intensity modulator is 45 microns. The sample support 4 E is rotatably mounted about an axis of rotation orthogonal to the main direction of the X-rays from the X-ray source 2. The X-ray detection system 5 is placed at a distance D equal to 530 mm from the X-ray source 2. The sample E is placed at a distance d equal to 25 mm from the X-ray source. The photo-detector elements 5a of the X-ray detection system 5 are square in shape and have a size of 127 μm. The average roughness of the spatial intensity modulator 3 is in this case equal to 7.5 times the size of the pixels of the first image I_{ref}(x, y).

The support 4 of sample E is placed in different positions, by successive rotations relative to the X-ray source 2. A two-dimensional image of the sample E is recorded by the detection system 5 for each defined position of the support 4. The set of the two-dimensional images of the fly allows reconstructing a three-dimensional volume rendering of the fly.

In FIG. 8, the image (a) represents a conventional radiography of the sample E obtained with a conventional X-ray source of the source type for X-ray microtomography, without the use of a spatial intensity modulator. The image (b) represents a two-dimensional image of the same fly obtained according to the second embodiment of the X-ray imaging method according to the invention, with the same relative position of the sample E relative to the X-ray source 2 used only for the image (a). The image (b) has much less noise than image (a) and is much more contrasted. The image (c) is a volume rendering image of the sample E obtained with the second embodiment of the X-ray imaging method according to the invention and by combining the different two-dimensional images corresponding to each of the positions of the support 4 which were obtained by rotation. A volume rendering image is a two-dimensional projection of a series of three-dimensional data.

### Example 5: Obtaining Elastographic Data

In this example, the third embodiment of the imaging method is implemented according to the invention to characterise the elasticity of a sample E consisting of an assembly formed by a latex glove, polystyrene and a wire rope.

The X-ray source 2 is herein a synchrotron type source. The spatial intensity modulator 3 consists of copper powder with an average particle size of 45 μm. The average roughness of this spatial intensity modulator is 45 microns. The X-ray detection system 5 is placed at a distance D equal to 156 m from the X-ray source 2. The sample E is placed at a distance d equal to 145 m from the X-ray source. The photo-detector elements 5a of the x-ray detection system 5 are square in shape and have a size of 23 microns. The average roughness of the spatial intensity modulator 3 is in this case equal to 2.1 times the size of the pixels of the first image I_{ref}(x, y). A sound wave of frequency 100 and 250 Hz, acting as an excitation wave, is triggered during a time interval T=1 min. This wave sets the sample E in motion. The time tracking of the speckles generated by the spatial intensity modulator 3 then allows reconstituting the field of the maximum displacement at any point of the sample E. In FIG. 9, the image (a) shows an image of the projection of the sample E at an instant t captured in 2 ms. The image (b) illustrates the maximum displacement of each point of the sample E. By numerical derivation of the maximum displacement, the value of the Young's modulus of the sample E at each of its points is obtained. The image (c) illustrates the value of the Young's modulus of each point in sample E.

### LIST OF REFERENCES

F. Pfeiffer, C. Kottler, O. Bunk, and C. David, Hard x-ray phase tomography with low-brilliance sources,” Physical Review Letters 98, 108105 (2007).
Alessandro Olivo and Robert Speller, A coded-aperture technique allowing x-ray phase contrast imaging with conventional sources, Applied Physics Letters 91, 074106{3 (2007)
E. Brun et al., High-resolution, low-dose phase contrast x-ray tomography for 3d diagnosis of human breast cancers, Proceedings of the National Academy of Sciences 109, 18290-4 (2012)
A. Snigirev, I. Snigireva, V. Kohn, S. Kuznetsov, I. Schelokov, On the possibilities of x-ray phase contrast microimaging by coherent high-energy synchrotron radiation, Rev. Sci. Instrum. 66 (12) December 1995.
K. A. Nugent, T. E. Gureyev, D. F. Cookson, D. Paganin, Z. Barnea, Quantitative Phase Imaging Using Hard X Rays, Phys. Rev. Letters, 0031-9007/96/77(14)/2961(1996).
C. Raven, A. Snigirev, I. Snigireva, P. Spanne, A. Suvorov, V. Kohn, Phase-contrast microtomography with coherent high-energy synchrotron x rays.
P. Cloetens, W. Ludwig, D. Van DYCK, J. P. Guigay, M. Schlenker; J. Baruchel, Quantitative phase tomography by holographic reconstruction, SPIE's International Symposium on Optical Science, Engineering, and Instrumentation, Proceedings Volume 3772, Developments in X-Ray Tomography II, 1999.
R. Cerbino, L. Peverini, M. A. C. Potenza, A. Robert, P. Bosecke, and M. Giglio, X ray-scattering information obtained from near-field speckle,” Nature Physics 4, 238-43 (2008).
Sebastien Berujon, Eric Ziegler, Roberto Cerbino, and Luca Peverini, Two-dimensional x-ray beam phase sensing, Physical Review Letters 108, 158102 (2012).
Kaye S. Morgan, David M. Paganin, and Karen K. W. Siu, X-ray phase imaging with a paper analyzer, Applied Physics Letters 100, 124102-4 (2012).
Sebastien Berujon, Hongchang Wang, Ian Pape, and Kawal Sawhney, X-ray phase microscopy using the speckle tracking technique, Applied Physics Letters 102, 154105-4 (2013).
Sebastien Berujon and Eric Ziegler, X-ray multimodal tomography using speckle vector tracking, Physical Review Applied 5, 044014 (2016).
U. Lundström, D. H. Larsson, A. Burvall, P. A. C. Takman, L. Scott, H. Brismar, H. M. Hertz, X-Ray phase contrast for CO2 microangiography, Phys. Med. Biol. 57 (2012) 2603-2617.
A. Momose, W. Yashiro, K. Kido et al., X-Ray phase imaging: from synchrotron to hospital, Phil. Trans. R. Soc. A 372: 20130023.
P. C. Diemoz, C. K. Hagen, M. Endrizzi et al., Single-Shot X-Ray Phase-Contrast Computed Tomography with Nonmicrofocal Laboratory Sources, Phys. Rev. Applied 7, 044029 (2017).
M.-C; Zdora, P. Thibault, T. Zhou et al., X-ray Phase-Contrast Imaging and Metrology through Unified Modulated Pattern Analysis, PRL 118, 203903 (2017).
D. M. Paganin, H. Labriet, E. Brun, S. Berujon, Single-image geometric-flow x-ray speckle tracking, Physics. med. (2018).

1. An X-ray imaging device, in particular in phase contrast, characterised in that said imaging device comprises:
an X-ray source,
a spatial intensity modulator, having a maximum thickness and a minimum thickness, capable of being passed through by an X-ray beam from the X-ray source, and of forming an X-ray beam which is spatially intensity modulated, the spatial intensity modulator comprising an element selected from copper, titanium, nickel, silver, tin, gold, and the mixtures thereof,
a support of sample (E), capable of supporting a sample (E), said sample (E) being intended to be passed through by at least one portion of said spatially intensity modulated X-ray beam and transmitting a refracted spatially intensity modulated X-ray beam, the sample (E), when it is on said support, being located at a distance d from the X-ray source,
an X-ray detection system, located at a distance D from the X-ray source, and comprising a two-dimensional X-ray sensor provided with a plurality of photo-detector elements, each having the same given size, said detection system being capable, in a first configuration of the device in which the device does not comprise a sample (E), of detecting a first X-ray beam F directly from the spatial intensity modulator, said first X-ray beam directly from the spatial intensity modulator having a first intensity modulation, and of transforming said first beam into a first electrical signal (Selec_ref), and, in a second configuration of the device in which a sample (E) is disposed on the support of sample (E), of detecting a second X-ray beam F′, passing through the spatial intensity modulator then the sample (E), said second X-ray beam refracted by the sample (E) having a second intensity modulation, and of transforming said second beam into a second electrical signal (Selec_sample),
an electronic processing unit, capable of receiving the first electrical signal (Selec_ref) and of processing it so as to generate a first image (Iref(x, y)) comprising a plurality of pixels, each having the same given size, and of receiving the second electrical signal (Selec_sample) and of processing it so as to generate a second image (Isample(x, y)), and capable of generating, from said first image (Iref(x, y)) and from said second image (Isample(x, y)), at least one characteristic image of said sample (E), said imaging device being characterised in that the difference between the maximum thickness and the minimum thickness of the spatial intensity modulator (3), called average roughness, is comprised between two and twenty times the size of the pixels of the first image Iref(x, y), said size being equal to the product of the size of the photo-detector elements by the ratio d/D.

2. The device according to claim 1, characterised in that the photo-detector elements are of the same square shape and each have the same given size equal to the length of the side of the square shape.

3. The device according to claim 1, characterised in that the photo-detector elements (5a) are of the same rectangular shape and each have the same given size equal to the length of the rectangular shape.

4. The device according to claim 1, characterised in that the electronic processing unit is capable of generating, from the difference between said first image (Iref(x, y)) and said second image Isample(x, y) at least one characteristic image of said sample (E).

5. The device according to claim 1, characterised in that the electronic processing unit is capable of generating said at least one characteristic image of said sample (E) depending on the gradient of the phase of the refracted X-ray beam F′ received by the detection system in the second configuration of the device.

6. The device according to claim 1, characterised in that the spatial intensity modulator comprises a material selected from a metal, a metalloid, a light element and the mixtures thereof, the atomic number of said element being comprised between 13 and 80.

7. The device according to claim 1, characterised in that the spatial intensity modulator comprises powder and/or particles.

8. The device according to claim 1, characterised in that the X-ray source is capable of emitting photons of energy comprised between 10 and 300 keV.

9. The device according to claim 1, characterised in that the X-ray source is selected from a source for computer-assisted microtomography, for example of the nanoFocus or microFocus type, a source for a brightness amplifier type apparatus, a source for a mammography type apparatus, and a radiography type source.

10. The device according to claim 1, characterised in that the support of the sample (E) is rotatably mounted about an axis of rotation orthogonal to the main direction of the X-ray beam from the X-ray source.

11. The device according to claim 1, characterised in that the assembly formed by the X-ray source and the X-ray detection system is rotatably mounted around the support of sample (E).

12. An X-ray imaging method, characterised in that it implements an X-ray imaging device according to claim 1.

13. The method according to claim 12, characterised in that it is a two-dimensional imaging method, said method comprising the following steps:
a/ exposing the spatial intensity modulator to an X-ray beam from the X-ray source,
b/ in the first configuration of the device, detecting and transforming the first X-ray beam F into a first electrical signal (Selec_ref),
c/ receiving and processing, by the electronic unit, the first electrical signal (Selec_ref), so as to generate at least one first image (Iref(x, y)),
d/ in the second configuration of the device, detecting and transforming the second X-ray beam F′ into a second electrical signal (Selec_sample),
e/ receiving and processing by the electronic processing unit the second electrical signal (Selec_ref), so as to generate at least one second image (Isample(x, y)),
f/ generating, by the electronic processing unit, from said at least one first image (Iref(x, y)) and said at least one second image (Isample(x, y)), at least one image selected from a transmission image, an image of the gradient of the differential phase in two directions which are orthogonal and parallel to a plane perpendicular to the main direction of the X-ray beam from the X-ray source, an image of the phase, and an image of the scattering of the sample (E).

14. The method according to claim 12, in the case where the support of the sample (E) is rotatably mounted about an axis of rotation orthogonal to the main direction of the X-ray beam from the X-ray source or where the assembly formed by the X-ray source and the X-ray detection system is rotatably mounted around the support of sample (E), characterised in that the method is a three-dimensional imaging method, said method comprising the following steps:
a/ exposing the spatial intensity modulator to an X-ray beam from the X-ray source,
b/ in the first configuration of the device, detecting and transforming the first X-ray beam F into a first electrical signal (Selec_ref),
c/ receiving and processing, by the electronic processing unit, the first electrical signal (Selec_ref), so as to generate at least one first image (Iref(x, y)),
d/ in the second configuration of the device, in the case where either the support of sample (E) is rotatably mounted about an axis of rotation orthogonal to the main direction of the X-ray beam from the X-ray source, or the assembly formed by the X-ray source and the X-ray detection system is rotatably mounted around the support of sample (E), detecting the second X-ray beam F′ and transforming it into a second electrical signal (Selec_sample_i), for N given positions (1, . . . , i, . . . , N), the N given positions being either positions of the sample (E), each corresponding to a given rotation of the support of sample (E), or positions of the assembly formed by the X-ray source and the X-ray detection system,
e/ receiving and processing by the electronic processing unit, for each of the N positions, the second electrical signal (Selec_sample-i), so as to generate for each of the N positions at least one second image (Isample_i(x, y)),
f/ generating, by the electronic processing unit, from said at least one first image (Iref(x, y)) and all second images (Isample-i(x, y)), at least one image selected from a three-dimensional transmission image, a three-dimensional image of the gradient of the differential phase in two directions which are orthogonal and parallel to a plane perpendicular to the main direction of the X-rays from the X-ray source, a three-dimensional image of the phase, and a three-dimensional image of the scattering of the sample (E).

15. The method according to claim 13, characterised in that a plurality of first images (Iref(x, y)) and a plurality of second images (Isample(x, y)) are generated and are combined to generate by the electronic processing unit at least one two-dimensional or three-dimensional image selected from a transmission image, an image of the gradient of the differential phase in two directions which are orthogonal and parallel to a plane perpendicular to the main direction of the X-ray beam from the X-ray source, an image of the phase, and an image of the scattering of the sample (E).

16. The method according to claim 12, characterised in that it is a method for time tracking structures contained in a sample (E), during a time interval T, said method comprising the following steps:
a/ exposing the spatial intensity modulator to an X-ray beam from the X-ray source,
b/ in the first configuration of the device, detecting and transforming the first X-ray beam F into a first electrical signal (Selec_ref),
c/ receiving the first electrical signal (Selec_ref) and processing it so as to generate at least one first image (Iref(x, y)),
d/ detecting, for a plurality of N instants ti, i being an integer comprised between 1 and N, contained in the time interval T, and transforming the second X-ray beam F′ into a plurality of N second electrical signals (Selec_sample_i), for i ranging from 1 to N,
e/ receiving and processing by the electronic processing unit the N second electrical signals (Selec_sample_i), for i ranging from 1 to N, so as to generate N second images (Isample (x, y)),
f/ generating, by the electronic processing unit, from said at least one first image (Iref(x, y)) and N second images (Isample_i(x, y)), a sequence of N successive images (Idef_i(x, y)), said N successive images (Idef_i(x, y)) being transmission images, gradient images of the differential phase in two directions which are orthogonal and parallel to a plane perpendicular to the main direction of the X-rays from the x-ray source, images of the phase, or images of the scattering of the sample (E).

17. The method according to claim 16, wherein a plurality of first images (Iref(x, y)) and a plurality of second images (Isample_i(x, y)) are generated and are combined to generate by the electronic processing unit at least one sequence of N successive images (Idef_i(x, y)), for i ranging from 1 to N, said N successive images (Idef_i(x, y)) being transmission images, gradient images of the differential phase in two directions which are orthogonal and parallel to a plane perpendicular to the main direction of the X-rays from the X-ray source, images of the phase, or images of the scattering of the sample (E).

18. The method according to claim 16, characterised in that it further comprises a step of processing said at least one sequence of N successive images (Idef_i(x, y)) to obtain elastographic data of the structures of the sample (E).
